(19) United States
(12) Patent Application Publication
(10) Pub. No.: US 2010/0106079 A1
(43) Pub. Date: Apr. 29, 2010

(54) METHOD AND APPARATUS FOR TREATING A WOUND

(75) Inventor: HOJABR ALIMI, Petaluma, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Oculus Innovative Sciences, Inc., Petaluma, CA (US)

(21) Appl. No.: 12/477,792

(22) Filed: Jun. 3, 2009

Related U.S. Application Data
(60) Provisional application No. 61/058,208, filed on Jun. 3, 2008.

(51) Int. Cl. A61M 35/00; A61L 15/16; A61K 33/00; A61M 37/00

(52) U.S. Cl. 604/24; 424/444; 424/722; 604/23; 604/289

(57) ABSTRACT

The present invention relates to a method of treating a wound and an apparatus for promoting wound healing. An enclosure is applied over the site of a wound and filled with a physiologically acceptable rinse solution. The physiologically acceptable rinse solution treats the wound and can promote the growth of tissue cells in the wound. Following exposure to the wound, the physiologically acceptable rinse solution is analyzed by a data sensor to determine the wound bed biology.

### CROSS-REFERENCE TO RELATED APPLICATIONS

This patent application claims the benefit of U.S. Provisional Patent Application No. 61/058,208, filed Jun. 3, 2008, which is incorporated by reference.

### FIELD OF THE INVENTION

The present invention relates to a method of treating a patient having a surface wound. The present invention is additionally directed to an apparatus for treating a patient having a surface wound.

### BACKGROUND OF THE INVENTION

Conventional wound treatment involves the use of wound coverings such as bandages, gauzes, packings, and dressings. Such coverings are generally employed to mechanically close and/or treat wounds and, as such, directly contact the patient's wounded tissue. Physical contact with the wound can interfere with the healing process and cause discomfort to the patient.

In addition, when treated in a traditional manner, there is often a need to remove and then replace the wound covering in an effort to determine the status of wound healing. The wound covering material must also be changed frequently because it can become saturated with effluent material discharged from the wound. The need to change the dressing can further increase when the caregiver applies treatment fluids to the wound covering (e.g., saline solution, peroxide, topical antibiotics, etc.).

Removing and/or changing a wound dressing exposes the wound to the open atmosphere and can increase the risk of infection. Additionally, this process can cause significant discomfort to the patient as well as further trauma to the wound. This is because conventional wound coverings can bind with, or stick to exposed tissue within the wound, thus causing a loss of tissue from the wound and pain for the patient. All of these factors contribute to a slowing of the healing process.

Thus, caregivers and patients would benefit from a wound treatment system that provides a wound covering that does not physically contact the wound, allows for disposal of material discharged from a wound in the course of treatment, and eliminates the need to remove the wound covering material until treatment is complete.

### BRIEF SUMMARY OF THE INVENTION

In view of the forgoing, the present invention provides a method of treating a patient suffering from a surface wound with an enclosure that does not come into contact with the wound itself. The method of the present invention is conducted with the following steps:

(a) an enclosure is applied to the site of a wound;

(b) a physiologically acceptable rinse solution which promotes the growth of tissue cells is then introduced and is held in direct contact with the wound by the enclosure; and

(c) a portion of the rinse solution is removed from the enclosure and analyzed to determine the wound bed biology, wherein the internal pressure within the enclosure is maintained during wound treatment such that the surface of the enclosure does not come into contact with the wound, and wherein no wound dressing is present within the enclosure.

The treatment method of the present invention is completed when the desired level of wound healing is obtained. Upon completion, the rinse solution can be drained from the enclosure and the enclosure can be removed from the patient.

The present invention additionally provides an apparatus for promoting wound healing in a patient. The apparatus of the present invention includes the following components:
(a) an enclosure having a continuous barrier layer with a peripheral edge that forms a seal with the surface of skin adjacent to a wound site;
(b) a positive pressure source which supplies a physiologically acceptable gas to the enclosure;
(c) a rinse solution source which supplies physiologically acceptable rinse solution to the enclosure; and
(d) a data sensor for analyzing one or more physical and/or characteristics of the physiologically acceptable rinse solution.

### BRIEF DESCRIPTION OF THE DRAWING

The present invention will be described with reference to the attached drawing by way of a non-limiting example only, in which:

FIG. 1 shows one embodiment of the wound healing apparatus of the present invention.

FIG. 2 shows a side view of the enclosure useful in the present invention.

### DETAILED DESCRIPTION OF THE INVENTION

The method and apparatus of the present invention are described as follows.

The present invention provides a method of treating a wound in a patient in need thereof comprising:
(a) applying an enclosure over a wound site;
(b) introducing a physiologically acceptable rinse solution into the enclosure to promote the growth of tissue cells, wherein the rinse solution is held in direct contact with the wound by the enclosure;
(c) removing at least a portion of the rinse solution from the enclosure;
(d) analyzing the rinse solution removed from the enclosure in step (c) to determine the wound bed biology; and
(e) draining the rinse solution from the enclosure when the desired wound bed biology is obtained based on the analysis in step (d);
   wherein the internal pressure within the enclosure is maintained during wound treatment such that the surface of the enclosure does not come into contact with the wound, and wherein no wound dressing is present within the enclosure.

The method of the present invention includes treatment of wounds on the outer surface of living tissue. This includes tissue which has been impaired or damaged by surgery. The method also includes tissue which has been impaired or damaged by causes that are not necessarily related to surgery, e.g., burns, cuts, abrasions, scrapes, rashes, ulcers, bed sores, puncture wounds, infections, or other lesions or breaks in the skin, and the like. Preferably, the method of the invention is used to treat second and third degree burns and diabetic skin ulcers. More preferably, diabetic foot ulcers can be treated by the method of the invention.

The enclosure of the present invention is a non-contact wound covering for controlling the local environment at a wound site on a patient. The enclosure protects the wound from contamination by materials from the outside environment and also prevents the wound site from shedding contaminants into the outside environment. The enclosure may not contain any wound dressings, such as bandages, gauzes, or packings, which come into direct contact with the wound. The fact the enclosure does not contact the wound itself promotes healing by minimizing mechanical stresses on the tissues.

In one embodiment, the surface of the wound is substantially free from contact by either the enclosure or wound dressings. Preferably, the surface of the enclosure does not come into contact with the wound.

The present invention is compatible with other technologies that can be used in the treatment of wounds. For example, skin grafts, Gore-Tex grafts, gels, creams, and/or pastes may be present on the surface of the wound within the enclosure.

It will be understood by those skilled in the art that the enclosure can be supplied in a variety of shapes and sizes to accommodate various wounds. Similarly, varying shapes and sizes can be employed so as to create a treatment volume over the wound site that will provide for optimal healing.

In one embodiment of the present invention, the enclosure is applied to an area of tissue fully encompassing the wound. In another embodiment, the enclosure is applied to an area of tissue partially encompassing the wound.

The enclosure comprises a continuous barrier layer of material having a peripheral edge that is applied to the skin surface adjacent to a wound site such that the edge forms a seal with the skin surface.

The barrier layer of the enclosure may be constructed of any suitable material. In a preferred embodiment, the barrier layer is flexible. The use of a flexible barrier layer allows for use on flexible joints such as elbows and knees. As an example, the barrier layer can be constructed of a flexible plastic film, such as polyethylene or polyvinylchloride. Alternatively, the barrier layer may be made of a semi-flexible or inflexible, rigid material.

The barrier layer may preferably be transparent to enable viewing the wound without removal. However, for cosmetic reasons the barrier layer may be opaque so that the wound cannot be seen through the enclosure.

In one embodiment, the barrier layer of the enclosure is substantially impermeable. More specifically, the barrier layer may be impermeable to the physiologically acceptable gas and the physiologically acceptable rinse solution employed in the present invention.

In another embodiment, the barrier layer of the enclosure is semi-permeable. In such a case, the barrier layer is preferably permeable to the physiologically acceptable gas but impermeable to the physiologically acceptable rinse solution. Alternatively, the barrier layer may be permeable to both the physiologically acceptable rinse solution and physiologically acceptable gas.

The peripheral edge of the barrier layer is applied to the skin surface to form a seal. It will be understood by those skilled in the art that the seal can be maintained using any suitable method. In one embodiment, a physiologically acceptable adhesive can be applied to the peripheral edge of the barrier to facilitate application of the enclosure to the patient's skin. However, with respect to comfort and efficiency, it may not always be desirable to use an adhesive on the patient's skin. Thus, in another embodiment of the present invention, the seal between the peripheral edge of the barrier layer and the patient's skin can be operatively maintained by the use of any suitable tape, strap, wrap, tab, or fastener.

The enclosure of the present invention can further comprise an internal support frame for the barrier layer. Such a support frame may be present to actively hold the barrier layer of the enclosure away from the wound. The internal support frame may be constructed out of any suitable material. Exemplary materials include plastic, foam or other foam-like materials. The support frame may be rigid or flexible. It will be understood by those skilled in the art that the support frame for the barrier layer can be supplied in a variety of shapes and sizes to accommodate various wounds. Similarly, varying shapes and sizes can be employed so as to create a treatment volume over the wound site that will provide for optimal healing.

The internal support frame may contact a portion of the wound site. Preferably, the wound is substantially free from contact by the support frame. More preferably, the internal support frame does not contact the wound site, but rests on the surface of the skin around the wound. An adhesive system may be used to attach the internal support to tissue such as, for example, tape or other fastener.

The barrier layer may be connected to the internal support frame by any suitable means such as, for example, an adhesive. Alternatively, the barrier layer may rest on the internal support without mechanical or chemical means.

The enclosure of the present invention may be pressurized with a physiologically acceptable gas. The gas is used to control the local atmosphere around the wound and to inflate the enclosure so as to hold the barrier layer of the enclosure away from the wound. In the abovementioned embodiment, wherein an internal support frame for the barrier layer is employed, the physiologically acceptable gas assists the support frame in holding the barrier layer off of the wound surface. The enclosure can be pressurized with any suitable physiologically acceptable gas. Preferably, the physiologically acceptable gas is oxygen, nitrogen, or ambient air.

The physiologically acceptable gas may be introduced into the enclosure by an external positive pressure source. The positive pressure source can be any suitable positive pressure source known in the art. Preferably, the positive pressure source may be a pressurized gas cylinder or a gas compressor. Illustratively, a diaphragm type gas compressor driven by motor may be used.

In one embodiment, the positive pressure source can continuously supply a physiologically acceptable gas to the enclosure. When continuously supplying a physiologically acceptable gas, the positive pressure source can be adjusted for any desired pressure and/or flow rate. In an alternative embodiment, the positive pressure source can intermittently supply a physiologically acceptable gas to the enclosure. In such a case, the positive pressure source can operate in a pulsed sequence that can be adjusted for any desired frequency, intensity, pressure, and/or flow rate.

In the method of the present invention, a physiologically acceptable rinse solution is introduced into the enclosure to promote the growth of tissue cells. The rinse solution is held in direct contact with the wound by the enclosure and inflates the enclosure so as to hold the barrier layer of the enclosure away from the wound.

Any suitable physiologically acceptable rinse solution may be employed. Examples of suitable physiologically acceptable rinse solutions include: pure, ultrapure, or deionized water, saline solution, peroxide, silver-based liquid, charged anode water, oxidative reductive potential water (also known as ORP water or super-oxidized water), or an electrolyzed solution.

The ORP water useful in the present invention can include various ionic and other species such that the growth of tissue cells is maximized. For example, the oxidative reductive potential water solution may contain chlorine (e.g., free chlorine and bound chlorine), active oxygen, ozone, and peroxides (e.g., hydrogen peroxide). Free chlorine typically includes, but is not limited to, hypochlorous acid (HClO), hypochlorite ions (ClO⁻), sodium hypochlorite (NaOCl), chloride ion (Cl⁻), chlorite ions (ClO₂⁻), chlorine dioxide (ClO₂), dissolved chlorine gas (Cl₂), and other radical chlorine species. The active oxygen can include singlet oxygen (¹O₂) formed by excitation of triplet oxygen, superoxide (O₂⁻) formed by reduction of oxygen by a single electron, and hydroxy radical (HO.), as well as hypochlorous ions (ClO⁻), and peroxy radical (ROO.), alkoxy radical (RO.), and hydroperoxide (ROOH) all formed by reactions with biobody ingredients such as unsaturated aliphatic acids (R). Preferably, the ORP water is stable for at least 24 hours and a pH in the range of about 6.0 to about 8.0.

Examples of a physiologically acceptable rinse solution are described in U.S. Patent Application Publication Nos. 2002/0160053 A1, 2005/0139808 A1, 2005/0196462 A1 and 2005/0142157 A1, all of which are incorporated herein by reference.

In another aspect, the physiologically acceptable rinse solution can contain dichlorine monoxide as described in International PCT Application No. PCT/US08/56919 (filed Mar. 13, 2008), incorporated herein by reference.

The physiologically acceptable rinse solution can be introduced into the enclosure either alone or in combination with one or more other therapeutic agents. Any suitable additional therapeutic can be used. Non-limiting examples of such therapeutics are antibiotics (e.g., levofloxacin) and local analgesics.

The physiologically acceptable rinse solution may be introduced into the enclosure by an external positive pressure source. The positive pressure source can be the same or different from the positive pressure source used to introduce the physiologically acceptable gas set forth above. The positive pressure source can be any suitable positive pressure source known in the art.

In one embodiment, the positive pressure source can continuously supply a physiologically acceptable rinse solution to the enclosure. When continuously supplying a physiologically acceptable rinse solution, the positive pressure source can be adjusted for any desired pressure and/or flow rate. In an alternative embodiment, the positive pressure source can intermittently supply a physiologically acceptable rinse solution to the enclosure. In such a case, the positive pressure source can operate in a pulsed sequence that can be adjusted for any desired frequency, intensity, pressure, and/or flow rate.

In the method of the invention, the enclosure avoids contact with substantially the entire surface of the wound. Preferably, the enclose does not contact with the surface of the wound. The enclosure may be kept away from the wound surface by any suitable means. For example, the enclosure may have a barrier layer made of a rigid, inflexible material. Alternatively, the enclosure may further comprise an internal support frame that keeps the barrier layer off the wound surface. In another embodiment, the pressure from the physiologically acceptable rinse solution, physiologically acceptable gas and combinations thereof may be used such that the enclosure does not contact the wound.

After the wound is exposed to the physiologically acceptable rinse solution, the entire physiologically acceptable rinse solution or a portion thereof can be removed from the enclosure to determine the wound bed biology. The wound bed biology is evaluated by analyzing one or more physical and/chemical characteristics of the rinse solution. Exemplary physical and chemical characteristics of the rinse solution include organic load, oxidative reductive potential, microbial load, pH, chlorine level, oxygen content, and combinations thereof. As a non-limiting example, organic load such as the natural macrophages, immunoprotective cells, transmission factors, and growth factors present during reconstruction of connective tissues can be monitored.

The wound bed biology is useful in determining the extent of wound healing or wound cleaning resulting from the application of the rinse solution. For example, the organic load present in the rinse solution may indicate that sufficient wound healing has occurred such that there is no need for further treatment.

In a further embodiment, after the wound is exposed to the physiologically acceptable rinse solution, a portion of the physiologically acceptable rinse solution can be removed from the enclosure to determine the remaining ability of the physiologically acceptable rinse solution to promote the growth of tissue cells. Remaining ability to promote the growth of tissue cells can be determined based on one or more physical and/or chemical characteristics of the physiologically acceptable rinse solution. Exemplary physical and/or chemical characteristics include organic load, oxidative reductive potential, microbial load, pH, chlorine level, oxygen content, and combinations thereof. As a non-limiting example, the amount of oxidative reductive potential can be monitored to determine the ability to promote the growth of tissue cells.

The rinse solution may be removed from the enclosure by any suitable means. In one embodiment, the rinse solution is removed by positive pressure using a pump and a physiologically acceptable gas (e.g., oxygen, nitrogen, air, etc.). In an alternative embodiment, the rinse solution is removed by negative pressure. A vacuum pump may be used to apply a negative pressure to the enclosure thereby removing the rinse solution.

In an additional embodiment, the method of treating a wound of the present invention further comprises an additional step after the physiologically acceptable rinse solution is analyzed. More specifically, additional physiologically acceptable rinse solution can be introduced into the enclosure and the removing and analyzing steps can then be repeated. The additional physiologically acceptable rinse solution can be new rinse solution, rinse solution recirculated after removing it from the enclosure for analysis, and combinations thereof. When the additional physiologically acceptable rinse solution is recirculated, it may be necessary to treat the additional physiologically acceptable rinse solution to remove at least a portion of the organic load. Organic load can be removed from the physiologically acceptable rinse solution by any suitable method known to those of skill in the art. Preferably, the organic load is removed with a sponge or filter.

The present invention further provides an apparatus for promoting wound healing in a patient comprising:
(a) an enclosure comprising a continuous barrier layer having an peripheral edge to form a seal with a skin surface adjacent to a wound site;
(b) a positive pressure source connectively attached to the enclosure for supplying a physiologically acceptable gas to the enclosure;
(c) a rinse solution source connectively attached to the enclosure for supplying physiologically acceptable rinse solution to the enclosure; and
(d) a data sensor for analyzing one or more physical and/or chemical characteristics of the physiologically acceptable rinse solution connectively attached to the enclosure.

The enclosure, barrier layer, and positive pressure source of the apparatus are all described above in connection with the method of the present invention.

The apparatus of the present invention can further comprise an inline sponge or filter positioned between the enclosure and the data sensor that reduces the organic load in the physiologically acceptable rinse solution circulated within the apparatus.

The data sensor is present in the apparatus to measure one or more physical and/or chemical characteristics of the physiologically acceptable rinse solution which has contacted the wound. As detailed above, such measurements are taken so as to determine the wound bed biology and/or the remaining ability of the physiologically acceptable rinse solution to promote the growth of tissue cells. Wound bed biology may indicate the extent of wound healing that has occurred. The data sensor can be any suitable sensor known to those of skill in the art to measure characteristics such as organic load, oxidative reductive potential, microbial load, pH, chlorine level, and oxygen content.

The positive pressure source, rinse solution source, and data sensor are connectively attached to the enclosure through a single channel or a series of channels. The channels employed in the apparatus can be any channels suitable to handle pressurized physiologically acceptable gas and physiologically acceptable rinse solutions. The channels are preferably non-kinking flexible hoses, tubes, or piping made from any suitable material and are of any suitable length.

In a further embodiment, the positive pressure source can supply the physiologically acceptable rinse solution from the rinse solution source to the enclosure through a channel. Similarly, the positive pressure source can supply the physiologically acceptable rinse solution to the data sensor through a channel.

In a preferred embodiment, the apparatus of the present invention comprises:
(a) an enclosure comprising a continuous semi-permeable barrier layer having an peripheral edge to form a seal with a skin surface adjacent to a wound site;
(b) a positive pressure source connectively attached to the enclosure through a first channel for supplying a physiologically acceptable gas to the enclosure;
(c) a rinse solution source connectively attached through the first channel to the enclosure for supplying physiologically acceptable rinse solution to the enclosure, wherein the positive pressure source supplies the physiologically acceptable rinse solution to the enclosure; and
(d) a data sensor for analyzing one or more physical and/or chemical characteristics of the physiologically acceptable rinse solution connectively attached to the enclosure through a second channel, wherein the positive pressure source removes the physiologically acceptable rinse solution from the enclosure.

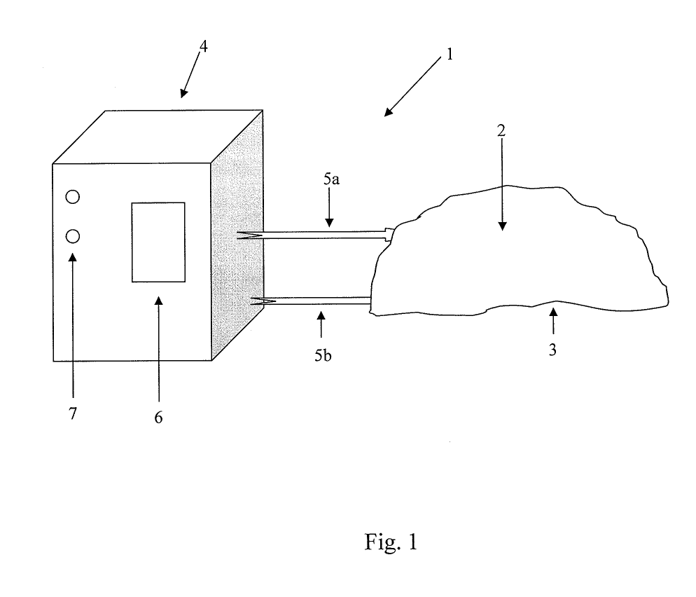

Turning now to FIG. 1, one embodiment of the apparatus 1 for promoting wound healing is shown. The enclosure 2 comprising a continuous barrier layer 3 is attached to a control housing 4 through a series of channels (5a and 5b). In this particular embodiment, the positive pressure source, the physiologically acceptable rinse solution source, and the data sensor are all contained within the housing 4. As drawn, the wound healing apparatus 1 further includes a computer-based electronic control system contained in the housing 4. The control system provides for automated control of the wound healing apparatus 1 for various treatment protocols. For example, the positive pressure source can be regulated by the control system to continuously or intermittently supply the physiologically acceptable rinse solution to the enclosure. The computer-based electronic control system further comprises a user interface 6 and indicator lights 7, thereby allowing the user to enter treatment protocols and receive feedback relating to the apparatus and the wound healing process.

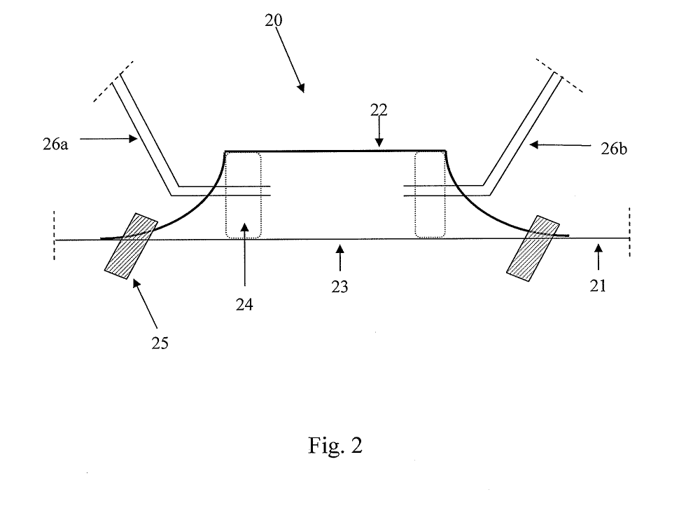

FIG. 2 further depicts another embodiment of the present invention wherein the enclosure 20 is shown attached to a patient's skin surface 21 in cross-section. The continuous barrier layer 22 is actively held away from the wound site 23 by an internal support 24. A seal between the peripheral edge of the continuous barrier layer 22 and the patient's skin is operatively maintained by medical tape 25. An input channel 26a is present to allow for supply physiologically acceptable gas and/or physiologically acceptable rinse solution to the wound site 23. In a similar manner, an output channel 26b is present to permit the removal physiologically acceptable gas and/or rinse solution from the wound site 23.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a wound in a patient in need thereof comprising:
(a) applying an enclosure over a wound site;
(b) introducing a physiologically acceptable rinse solution into the enclosure to promote the growth of tissue cells, wherein the rinse solution is held in direct contact with the wound by the enclosure;
(c) removing at least a portion of the rinse solution from the enclosure;
(d) analyzing the rinse solution removed from the enclosure in step (c) to determine the wound bed biology; and
(e) draining the rinse solution from the enclosure when the desired wound bed biology is obtained based on the analysis in step (d);
wherein the internal pressure within the enclosure is maintained during wound treatment such that the surface of the enclosure does not come into contact with the wound, and wherein no wound dressing is present within the enclosure.

2. The method of claim 1, wherein the physiologically acceptable rinse solution is an oxidative reductive potential water solution.

3. The method of claim 1, where in the physiologically acceptable rinse solution contains dichlorine monoxide.

4. The method of claim 1, wherein the wound bed biology in step (d) is determined based on one or more physical and/or chemical characteristics of the rinse solution selected from the group consisting of organic load, oxidative reductive potential, microbial load, pH, chlorine level, oxygen content, and combinations thereof.

5. The method of claim 4, further comprising step (d′) after step (d) of introducing additional physiologically acceptable rinse solution into the enclosure and repeating steps (c) and (d).

6. The method of claim 5, wherein the additional physiologically acceptable rinse solution is selected from the group consisting of new rinse solution, rinse solution recirculated from step (c), and combinations thereof.

7. The method of claim 5, wherein the additional physiologically acceptable rinse solution is rinse solution recirculated from step (c) that has been treated to remove at least a portion of the organic load.

8. The method of claim 7, wherein the organic load is removed with a sponge or filter.

9. The method of claim 1, wherein the enclosure comprises a continuous barrier layer of flexible material having a peripheral edge that is applied to the skin surface adjacent to a wound site such that the edge forms a seal with the skin surface.

10. The method of claim 9, wherein the barrier layer is impermeable.

11. The method of claim 9, wherein the barrier layer is semi-permeable.

12. The method of claim 9, wherein the enclosure further comprises an internal support frame for the barrier layer.

13. The method of claim 1, further comprising introducing a physiologically acceptable gas into the enclosure.

14. The method of claim 13, wherein the physiologically acceptable gas is introduced into the enclosure prior to step (b).

15. The method of claim 13, wherein the physiologically acceptable gas is introduced into the enclosure after step (b).

16. An apparatus for promoting wound healing in a patient comprising:
(a) an enclosure comprising a continuous barrier layer having an peripheral edge to form a seal with a skin surface adjacent to a wound site;
(b) a positive pressure source connectively attached to the enclosure for supplying a physiologically acceptable gas to the enclosure;
(c) a rinse solution source connectively attached to the enclosure for supplying physiologically acceptable rinse solution to the enclosure; and
(d) a data sensor for analyzing one or more physical and/or chemical characteristics of the physiologically acceptable rinse solution connectively attached to the enclosure.

17. The apparatus of claim 16, wherein the barrier layer is impermeable to the physiologically acceptable gas and the physiologically acceptable rinse solution.

18. The apparatus of claim 16, wherein the barrier layer is semi-permeable to the physiologically acceptable gas and the physiologically acceptable rinse solution.

19. The apparatus of claim 16, wherein the enclosure further comprises an internal support frame for the barrier layer.

20. The apparatus of claim 16, wherein the positive pressure source, rinse solution source and data sensor are connectively attached to the enclosure through a single channel or a series of channels.

21. The apparatus of claim 20, wherein the positive pressure source continuously supplies a physiologically acceptable gas to the enclosure.

22. The apparatus of claim 20, wherein the positive pressure source intermittently supplies a physiologically acceptable gas to the enclosure.

23. The apparatus of claim 20, wherein the positive pressure source supplies the physiologically acceptable rinse solution from the rinse solution source to the enclosure.

24. The apparatus of claim 23, wherein the positive pressure source supplies the physiologically acceptable rinse solution to the data sensor.

25. The apparatus of claim 24, further comprising an inline sponge or filter positioned between the enclosure and the data sensor that reduces the microbial load in the physiologically acceptable rinse solution that is removed from the enclosure.

26. An apparatus for promoting wound healing in a patient comprising:
(a) an enclosure comprising a continuous semi-permeable barrier layer having an peripheral edge to form a seal with a skin surface adjacent to a wound site;
(b) a positive pressure source connectively attached to the enclosure through a first channel;
(c) a rinse solution source connectively attached through the first channel to the enclosure for supplying physiologically acceptable rinse solution to the enclosure, wherein the positive pressure source supplies the physiologically acceptable rinse solution to the enclosure; and
(d) a data sensor for analyzing one or more physical and/or chemical characteristics of the physiologically acceptable rinse solution connectively attached to the enclosure through a second channel, wherein the positive pressure source removes the physiologically acceptable rinse solution from the enclosure.

27. The apparatus of claim 26, wherein the positive pressure source is capable of supplying a physiologically acceptable gas to the enclosure.
